(19) United States
(12) Patent Application Publication
(10) Pub. No.: US 2016/0158485 A1
(43) Pub. Date: Jun. 9, 2016

(54) HEAD MOUNTED LIGHT THERAPY DEVICE

(71) Applicant: Yumalite, Inc., Collingwood (CA)

(72) Inventors: Adam Givertz, Toronto (CA); Lee Fletcher, Toronto (CA); Sean Miller, Toronto (CA); Terence Woodside, Toronto (CA)

(21) Appl. No.: 14/793,812

(22) Filed: Jul. 8, 2015

Related U.S. Application Data
(63) Continuation of application No. 13/876,688, filed on Oct. 18, 2013, now abandoned.
(60) Provisional application No. 61/387,959, filed on Sep. 29, 2010.

(51) Int. Cl. A61M 21/00; A61N 5/06

(52) U.S. Cl. CPC A61M 21/00; A61N 5/0618; A61N 2005/0648; A61N 2005/0651; A61N 2005/0663; A61M 2021/0044

(57) ABSTRACT

A portable, lightweight, head-mounted light therapy device is disclosed. The device comprises an one or more selectable light sources, such as light emitting diodes (LEDs), to provide bright or dim red light, bright or dim white light, or a combination thereof, selectable by the user, for shining on the user's face and eyes. The device can be used for the treatment of light deficiency disorders or conditions and circadian rhythm disorders and disturbances.

### RELATED APPLICATION

This application claims the benefit of and priority to U.S. provisional patent application No. 61/387959, filed Sep. 29, 2010, which is incorporated herein in its entirety as though set forth explicitly herein.

### FIELD OF THE INVENTION

The present invention relates to light therapy devices. More particularly, the present invention relates to light therapy devices for the treatment of illness and diseases, such as seasonal affective disorder, winter blues and other light deficient disorders, and for the treatment of circadian rhythm disorders and disturbances.

### BACKGROUND

Seasonal change can result in low mood and energy. Light is responsible for establishing our circadian rhythms: biological patterns over a 24 hour period or what is generally known as a “biological clock”. Melatonin, a hormone produced by the pineal gland in humans is responsible for the entrainment of circadian rhythms for many biological functions. This naturally occurring hormone is only produced in humans at night and is therefore known as the ‘hormone of darkness’. Hormone production is, in fact, controlled according to light exposure. A type of cell in the retina, called photosensitive ganglion cells, interprets light/dark information and carries this information to the brain signalling the release of melatonin when dark. Melatonin secretion in humans can control a number of biological functions including reproduction, behaviour, hair growth, increases in leptin production (thereby increasing food intake), sleepiness, and libido suppression.

Another hormone, serotonin, also has a significant impact on mood and mood regulation and plays a major role in disruptions to circadian rhythms and in turn, mood regulation. Serotonin is converted into melatonin. This happens as light levels fall in the evening and the pineal gland signals for melatonin production to increase. Thus, the actions of serotonin and melatonin appear to be opposing, with serotonin stimulating us during the daytime and melatonin inducing sleep at night. In mood disorders caused by disruptions to circadian rhythms, melatonin levels tend to be higher than normal during the day; thus, sufferers experience sleepiness and other melatonin induced effects, and are also prone to serotonin deficiency symptoms such as negative emotional states. As a result of the close relationship between serotonin and melatonin, circadian rhythm dysfunction may be seen as complementary to each other rather than as distinct explanations for disorders caused by circadian rhythm disruptions or other light deficiencies.

Light deficiency is implicated in a variety of mood disorders including: Seasonal Affective Disorder (“SAD”), Sub-Syndromal Seasonal Defective Disorder, the “winter blues”, jet-lag, and shift work, the symptoms of which range from mild to major depression, lethargy, restlessness, increased appetite, diminished libido, weight gain, and general malaise. It has also been suggested that light deficiency can be implicated in more significant mood disorders including bi-polarism and major depressive syndromes.

The use of bright or dim red light, or bright or dim white light, directed toward a user's face and eyes may temporarily suppress brain melatonin levels and enhance brain serotonin, resulting in mood elevation and wakefulness. Further, the use of bright or dim red light, or bright or dim white light, directed toward a user's face and eyes may elevate the mood of users suffering from winter blues and is an effective treatment for other light deficient disorders, including SAD and Sub-Syndromal Seasonal Affective Disorder (S-SAD). Bright or dim red light, or bright or dim white light, directed toward a user's face and eyes may also be effective in the treatment of circadian rhythm disorders and disturbances, including jet-lag, sleep disorders and pre-menstrual syndrome (PMS) and has positive effects on weight loss, reduces fatigue and promotes wakefulness, and may also be effective in the treatment of certain sexual dysfunctions and chronic fatigue syndrome as well as in the treatment of non-seasonal depression and other psychiatric disorders, including antepartum depression, post-partum depression, bipolar disorder and major depressive disorder.

Lam, et al. (“The Can-SAD Study: A randomized controlled trial of the effectiveness of light therapy and fluoxetine in patients with winter seasonal affective disorder.” Am. J. Psychiatry, 2006;163:805-812) compared efficacy of light therapy versus fluoxetine treatments with patients with SAD and showed overall improvement with time, with no differences between the treatments, and supports the effectiveness and tolerability of both treatments for seasonal affective disorder.

In Gallin, et al. (“Ophthalmologic examination of patients with seasonal affective disorder, before and after bright light therapy.” Am J. of Ophthalmology 1995;119:202-210), patients suffering from SAD receiving light treatment showed no ocular changes or abnormalities following short or long term treatment, and showed that light therapy yields about a 75% clinical remissions rate.

Other studies have shown the benefits of dim red light versus bright red light (Levitt et al., “Dim versus bright red (light-emitting diode) light in the treatment of seasonal affective disorder.” Acta Psychiatr Scand 1994;89:341-45); treatment of SAD (Wileman et al., “Light therapy for seasonal affective disorder in primary care.” British J Psychiatry 2001;178:311-16);

Light therapy directed at the face and eyes of a user can be delivered via a variety of device designs employing various light sources, including fluorescent, halogen, laser, incandescent, or light-emitting diodes (LEDs). Desk and floor mounted light boxes were traditionally used to deliver bright white light to a user sitting in front of or facing the device. Such devices are often large, heavy and not easily portable. For example, U.S. Pat. No. 7,846,084 to McNew, describes a device having a compartment in an enclosure therein comprising a platform on which a user reclines his or her entire body. Devices of this sort are not practical for most patients, especially those for whom mobility is an issue. One benefit of a head mounted device is that it can deliver bright or dim red light or bright or dim white light with less restriction on a user's mobility.

In Meesters, et al. (“Prophylactic treatment of seasonal affective disorder (SAD) by using light visors: bright white or infrared light?” Biol. Psychiatry 1999;46:239-46), a study of 38 patients with SAD found that light visors can be effectively used to prevent the development of SAD.

Joffe et al. (“Light visor treatment for seasonal affective disorder: a multicenter study.” Psychiatry Research 1993;46:29-39) showed that light therapy using light visors has an antidepressant action by a nonspecific effect or that light is biologically active in the treatment of SAD across a wide range of intensities.

However, many currently available head-mounted devices rely on mounting mechanisms that are impractical and bulky, or are positioned too close to the eyes for comfort or convenience, and/or are not adjustable. Also, certain existing head mounted devices typically offer red, white or blue and green light, which may be inefficient for offering optimal treatment and comfort to the user.

U.S. Pat. No. 6,875,225, to Pederson et al. (The Litebook Company Ltd.), describes a hand-held light therapy device which generates a plurality of LEDs at 2,500 to 7,500 lux at 12 inches.

U.S. Pat. No. 7,850,720, to Shefi et al., describes a light therapy device. The device comprises a light source and mount means, including a headgear. The device is connected to a controller circuit and cooling fan. However, the device is not particularly suited for portable use, in bulky, and is not attractive to the wearer for everyday use. PCT Patent Publication WO 2010/092501, to Paul et al. (Koninklijke Philips Electronics, N.V.), describes a light therapy device comprising a plurality of lighting modules which attach to the user's face, surrounding the user's eyes. The use of adhesives to attach the lighting modules can be unpleasant to the user, particularly when applying or removing the lighting modules from the face on the delicate tissue surrounding the eyes.

What is needed, therefore, is a lightweight, adjustable and highly portable head mounted light box that delivers an array of any of bright or dim red light, bright or dim white light or a combination of bright or dim red and white light to a user's face and eyes, for treating various diseases or conditions associated with light deficiency.

This background information is provided for the purpose of making known information believed by the applicant to be of possible relevance to the present invention. No admission is necessarily intended, nor should be construed, that any of the preceding information constitutes prior art against the present invention.

### SUMMARY

An object of the present invention is to provide a portable, lightweight, head-mounted light therapy device.

In accordance with one aspect of the present invention, there is provided a portable head-mounted light therapy device comprising: a strap for securing the device to a user's head; and a light box connected to the strap and comprising one or more light sources selectable by the user and directable to a face or one or more eyes of the user, wherein the one or more light sources provides white light, red light, or a combination thereof.

In accordance with another aspect of the present invention, there is provided a portable head-mounted light therapy device comprising: a strap for securing the device to a user's head; and a light box connected to the strap and comprising one or more light sources selectable by the user and directable to a face or one or more eyes of the user, wherein the one or more light sources provides a combination of white light and red light.

In accordance with another aspect of the present invention, there is provided a light box for a portable head-mounted light therapy device, the light box comprising one or more light sources selectable by a user and directable to a face or one or more eyes of the user, wherein the one or more light sources provides white light, red light, or a combination thereof.

In accordance with another aspect of the present invention, there is provided a light box for a portable head-mounted light therapy device, the light box comprising one or more light sources selectable by a user and directable to a face or one or more eyes of the user, wherein the one or more light sources provides a combination of white light and red light.

The device in accordance with the present invention is an adjustable, head-mounted light therapy device, such as a visor or the like. Ideally, the device is portable and constructed from lightweight and durable materials. The device uses an array of bright or dim red light-emitting diodes (LEDs) and bright or dim white LEDs as a light source. The device of the present invention can have a luminous emittance configured to about 80 to about 800 lux at 5 cm. In one embodiment, the device is configured for red light at about 80 lux (at 5 cm) and white light at about 800 lux (at 5 cm). However, the device can be adjusted and/or reconfigured to change the light source output, such as with inclusion of additional LEDs, increased brightness of the LEDs, or an increase in power. The light sources can be selectable and switchable, either independently or in combination, such that one or more bright and/or dim red lights, and/or one or more bright or dim white lights, or a combination of bright and/or dim red and white lights, are activated at any given time. Thus, any combination of red and/or white lights can be illuminated. In one embodiment, the device provides both red lights and white lights.

In one embodiment, the device is configured with a resistor, or dimmer, that allows the user to increase or decrease the power to the LEDs, thereby increasing or decreasing (dimming) the light emitted by the device. LEDs provide a lightweight, energy efficient, inexpensive, and lasting light source. The LEDs are contained in an adjustable, tilting light box assembly that allows the user to determine optimal light direction for treatment, and adjust over eyeglasses if required. The array of light is directed through a diffuser lens that casts a broad wash of red light, white light or a combination of red and white light across the user's eyes for optimal treatment and comfort. The light box assembly contains one or more actuators that allows a user to select bright and/or dim red light, bright and/or white light or a combination of red and white light, and/or to switch the device on or off.

The light box assembly of the device of the present invention can be powered by any suitable means and, ideally, an inexpensive, portable, and readily available power source. In one embodiment, the present device contains three AAA dry-cell batteries.

The present device can also comprise an adjustable head mount assembly allowing users to adjust the fit of the device for optimal treatment and comfort.

The present device can fold to a form factor similar to folded glasses rendering it portable and convenient.

The present device provides an unexpected benefit over current light therapy devices, including existing floor and desk mounted devices and existing head mounted devices, by allowing a user to select either one or more bright or dim red lights, one or more bright or dim white lights or a combination of bright and/or dim red light and white light, in a lightweight, portable head mounted device.

In accordance with another aspect of the present invention, there is provided a method of treating a disease or condition associated with light deficiency in a user, comprising providing the portable head-mounted light therapy device of the present invention to the user, placing the device on the head of the user, actuating the light source and directing light from the light source to a face and one or more eyes of the user. The device of the present invention can be used for treating diseases or conditions such as seasonal affective disorder, sub-syndromal seasonal defective disorder, sub-syndromal seasonal affective disorder, jet-lag, generalized mood disorder, pre-menstrual syndrome, sleep disorder, or antepartum depression, post-partum depression, bipolar disorder, or major depressive disorder.

### BRIEF DESCRIPTION OF THE FIGURES

FIG. 1 is a perspective drawing of one embodiment of a head-mounted light therapy device of the present invention.

FIG. 2 is a perspective drawing of one embodiment of a light box assembly of the present invention.

FIG. 3 is a perspective drawing of one embodiment of a head mount assembly of the present invention.

FIG. 4 is a side view of one embodiment of a head-mounted light therapy device of the present invention.

### DETAILED DESCRIPTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s) and/or component(s) as appropriate.

In accordance with one aspect of the present invention there is provided a portable head-mounted light therapy device comprising: a strap for securing the device to a user's head; and a light box connected to the strap and comprising one or more light sources selectable by the user and directable to a face or one or more eyes of the user, wherein the one or more light sources provides white light, red light, or a combination thereof.

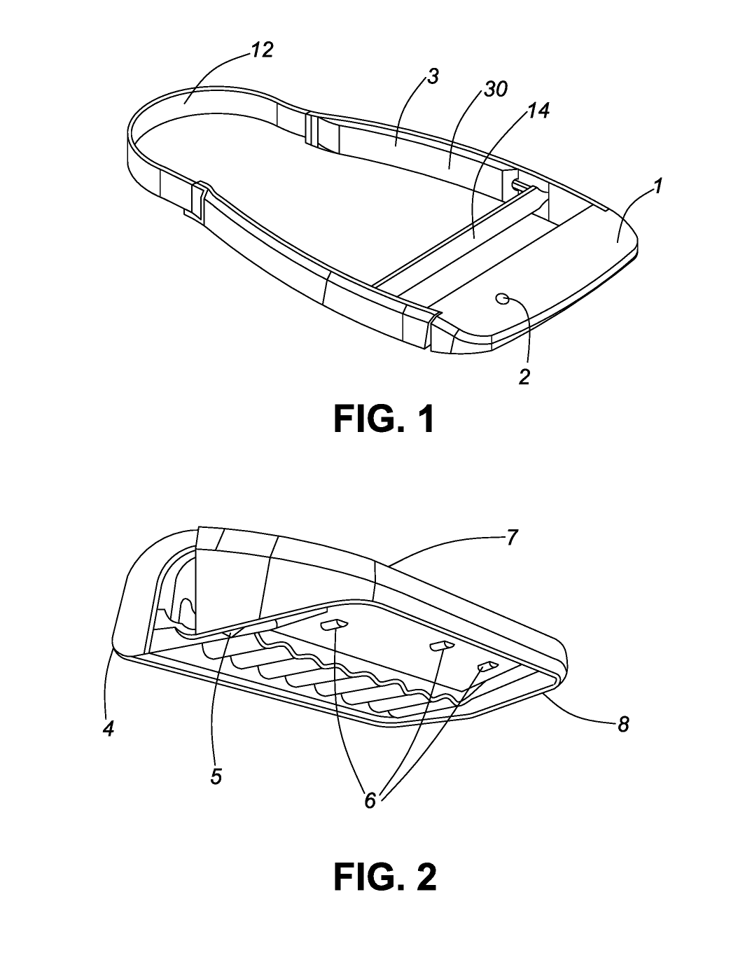

Referring to the figures, FIG. 1 shows one embodiment of the device of the present invention. Light box assembly 1 provides one or more light sources, such as wavelengths of white and red light, to the user's face and eyes. On the light box assembly 1, an actuator, such as a switching mechanism 2 is provided. The switching mechanism 2 can provide a number of options for the user. For example, activating the switching mechanism 2 can change the desired light source. This can include switching the light source from red light to white light, or to a combination of red and white light. Thus, the light sources can be switchable, either alone or in combination, such that one or more red and/or one or more white lights can be activated at any given time, to provide any combination of red and/or white lights, including both red lights and white lights. The switching mechanism 2 can also be used to turn on or off the light source. Alternatively, an additional actuator may be provided to activate or deactivate the light source.

The device of the present invention can have a luminous emittance configured to about 80 to about 800 lux at 5 cm. In one embodiment, the device is configured for red light at about 80 lux (at 5 cm) and white light at about 800 lux (at 5 cm). However, the device can be adjusted and/or reconfigured to change the light source output, such as with inclusion of additional LEDs, increased brightness of the LEDs, or an increase in power.

In one embodiment, the device is configured with a resistor, or dimmer (not shown), that allows the user to increase or decrease the power to the LEDs, thereby increasing or decreasing (dimming) the light emitted by the device. This can be in addition to the switching mechanism, or the dimming function can be part of the switching mechanism. As desired, the dimmer can cause one or more LEDs to dim, either of the same colour (for example, dimming all red lights or all white lights) or of a different colour (for example, dimming one or more red lights and/or one or more white lights).

As would be understood, the terms “bright” and “dim” intend to qualitatively describe the intensity of the light source. A “bright” light would have a higher luminous intensity than a “dim” light.

As shown in FIG. 1, the device of the present invention can have a head mount assembly 3. The head mount assembly 3 comprises an adjustable strap 30 which can sized to fit the dimensions of a user's head. The device can also sit on or above the ears of the user, to allow eyeglasses to be worn in conjunction with the device, if desired.

FIG. 2 shows one embodiment of the light box assembly in isolation. The light box assembly as shown comprises a generally rectangular housing 4 having a top cover 7. If desired, the top cover 7 can be embellished with fashionable colours or ornamental designs. The assembly comprises a circuit board 5 for controlling the light source. One or more LEDs 6 are provided. In typical embodiments, 1, 2, 3, 4, 5, 6 or more LEDs can be used. As mentioned above, one or more of the LEDs can be activated to provide a desired light source.

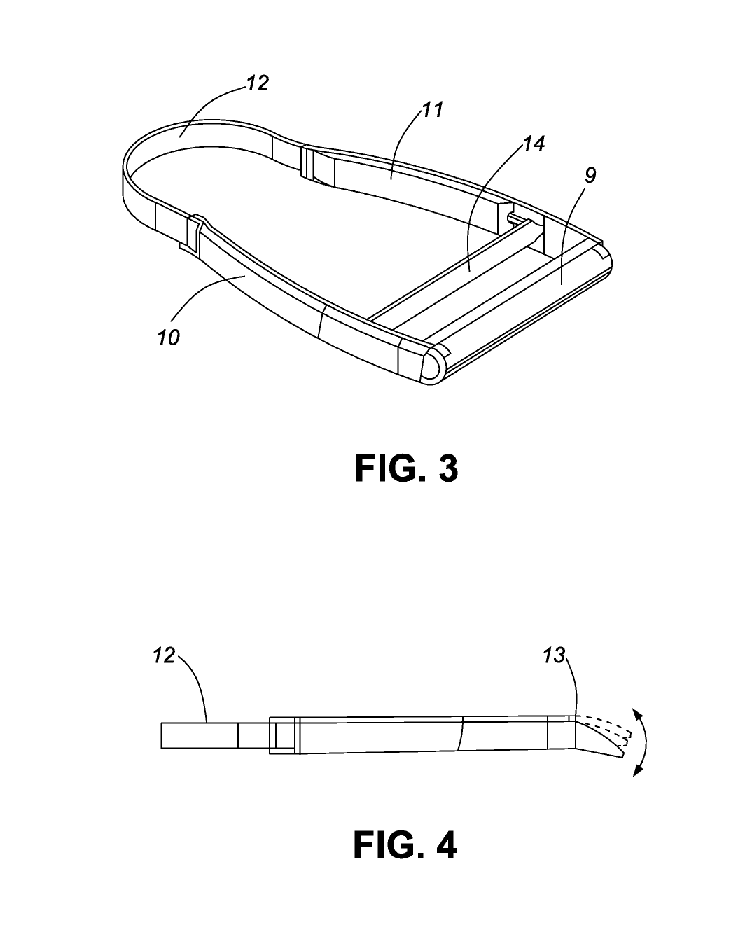

A lens 8 of a generally transparent material (such as, for example, plastic, glass or the like) can be present. The lens 8 protects the LEDs from damage and can provide a broad wash of light emanating from the light source to the user. Referring now to FIG. 3, there is shown one embodiment of a head mount assembly in isolation, which can be used in the device of the present invention. The head mount assembly generally comprises arms 10 and 11 which, when placed on a user, fit on each side of the user's head. The arms 10, 11 comprise a soft, resilient material, such as rubber, felt, plastic, or the like, to contour to the user's head and provide optimal comfort. Extending rearwardly from the arms is a strap, such as a backstrap 12 which fits on the back of the user's head. The backstrap 12 is typically of an elastic material to facilitate stretching and a snug fit on the user's head. The backstrap 12 can be integral with the arms 10, 11, or can be attached thereto. The arms 10, 11 and/or the backstrap 12 are adjustable in length using any standard lengthening mechanism known in the art.

The head mount assembly comprises a cross housing 9 to which arms 10, 11 connect. The cross housing 9 is generally perpendicular to the arms 10, 11, providing a base with which the light box assembly engages. The cross housing 9 typically contains the power source (such as batteries) for operating the light source in the light box assembly. In certain embodiments, arms 10, 11 connect with the cross housing at a hinge to provide additional flexibility to the arms. A cross strap 14 (which can either be integral with strap 12 or the material in arms 10, 11) can be provided. The cross strap 14 is generally parallel to the cross housing 9. The cross strap 14 is similarly of an elastic material to rest comfortably and snugly on the forehead of the user.

FIG. 4 shows one embodiment of the device of the present invention, as assembled with a light box assembly connected to a head mount assembly 13. The light box assembly can be affixed to or integral with the head mount assembly in any suitable way, and in some embodiments can be removable by the user. The light box assembly can be generally pivotable upwards or downwards relative to the head mount assembly 13. This allows the light box assembly, and the light emanating from it, to be tilted at an angle from a substantially vertical plane in front of and parallel to the face of the user. The light box assembly can be tilted ideally up to 25 degrees or more as required. For example, the light box assembly can be tilted about −5 degrees off the vertical plane (i.e., away from the user's face), or about +20 degrees off the vertical plane (i.e., toward the user's face). Alternatively, the entire device can be tilted up or down on the head by adjusting the strap (either the cross strap 14 or backstrap 12) and/or arms 10, 11.

In use, a user places the device of the present invention on top of his or her head, with the backstrap 12 at the back of the head and the cross strap 14 at the front. The user typically adjusts the backstrap 12 to ensure a snug fit of the device on his or her head. The user then activates the switching mechanism 2 to turn on the light source. The user can then activate the switching mechanism one or more additional times to select a desired light source, such as red light, white light, or a combination thereof, Alternatively, the user can tilt the angle of the entire device and/or the light box to provide a different light output at a different distance, if desired. The device can be kept activated, with one or more different light sources, for as long as desired by the user or as required by a practitioner.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A portable head-mounted light therapy device comprising:
a strap for securing the device to a user's head; and
a light box connected to the strap and comprising one or more light sources selectable by
the user and directable to a face or one or more eyes of the user, wherein the one or more light
sources provides white light, red light, or a combination thereof, and wherein the one or more light sources are switchable either alone or in combination to provide one or more white lights, one or more red lights, or a combination of white and red lights.

2. The device of claim 1, wherein the one or more light sources are one or more light emitting diodes (LEDs).

3. The device of claim 1, wherein the white light or red light is bright, dim, or a combination thereof.

4. (canceled)

5. The device of claim 1, wherein the strap is adjustable.

6. The device of claim 1, wherein the light box is pivotable at an angle relative to a substantially vertical plane to direct the one or more light sources at the angle to the user's face.

7. The device of claim 6, wherein the angle is from about −5 to about +20 degrees.

8. The device of claim 1, wherein the light box comprises an actuator for activating or adjusting the one or more light sources.

9. The device of claim 8, wherein the actuator is actuated in one or more positions to select the one or more light sources.

10. The device of claim 1, wherein the red light is configured to about 80 lux at 5 cm, and the white light is configured to about 800 lux at 5 cm.

11. A light box for a portable head-mounted light therapy device, the light box comprising one or more light sources selectable by a user and directable to a face or one or more eyes of the user, wherein the one or more light sources provides white light, red light, or a combination thereof, and wherein the one or more light sources are switchable either alone or in combination to provide one or more white lights, one or more red lights, or a combination of white and red lights.

12. The light box of claim 11, wherein the one or more light sources are one or more light emitting diodes (LEDs).

13. The light box of claim 11, wherein the white light or red light is bright, dim, or a combination thereof.

14. The light box of claim 10, wherein the one or more light sources provide a combination of white light and red light.

15. The light box of claim 10, wherein the red light is configured to about 80 lux at 5 cm, and the white light is configured to about 800 lux at 5 cm.

16-37. (canceled)

38. A method of treating a disease or condition associated with light deficiency or a circadian rhythm disorder or disturbance in a user, comprising providing the portable head-mounted light therapy device of claim 1 to the user, placing the device on the head of the user, actuating the light source and directing light from the light source to a face and one or more eyes of the user.

39. The method of claim 38, wherein the disease or condition associated with light deficiency is seasonal affective disorder, sub-syndromal seasonal defective disorder, sub-syndromal seasonal affective disorder, jet-lag, generalized mood disorder, pre-menstrual syndrome, sleep disorder, or antepartum depression, post-partum depression, bipolar disorder, or major depressive disorder.

40. The method of claim 38, wherein the disease or condition associated with light deficiency is depression.

41. In a portable head-mounted light therapy device for treating a disease or condition associated with light deficiency, the improvement comprising a means to increase efficacy of treatment of said disease or condition through the selection of white light, red light, or a combination thereof.

42-44. (canceled)
